(19) United States
(12) Patent Application Publication
(10) Pub. No.: US 2026/0073600 A1
(43) Pub. Date: Mar. 12, 2026

(54) X-RAY IMAGE DIAGNOSTIC APPARATUS, MEDICAL INFORMATION PROCESSING APPARATUS, MEDICAL INFORMATION PROCESSING SYSTEM, AND MEDICAL INFORMATION PROCESSING METHOD

(71) Applicant: CANON MEDICAL SYSTEMS CORPORATION, Otawara-shi (JP)

(72) Inventors: Asateru KIMURA, Yokohama (JP); Kosuke ARITA, Kawasaki (JP); Takuo NEGISHI, Nasushiobara (JP); Atsuko SUGIYAMA, Ota (JP)

(73) Assignee: CANON MEDICAL SYSTEMS CORPORATION, Otawara-shi (JP)

(21) Appl. No.: 19/323,499

(22) Filed: Sep. 9, 2025

(30) Foreign Application Priority Data
Sep. 10, 2024 (JP) 2024-156789

(51) Int. Cl. G06T 11/00; A61B 6/46; G06T 7/00; G16H 50/20

(52) U.S. Cl. CPC G06T 12/20; A61B 6/465; G06T 7/0016; G16H 50/20; G06T 2200/24; G06T 2207/10116; G06T 2207/30004; G06T 2210/41

(57) ABSTRACT

An X-ray image diagnostic apparatus or a medical information processing apparatus according to an embodiment includes an X-ray irradiator that outputs an X-ray and an X-ray detector that detects the X-ray output by the X-ray irradiator and transmitted through a subject. The X-ray image diagnostic apparatus or the medical information processing apparatus that generates an X-ray image based on detection data obtained by detection by the X-ray detector includes processing circuitry. The processing circuitry receives a first instruction, receive an instruction to add the X-ray image, acquires the degree of conformity between the first instruction and a second instruction based on a database in which a plurality of the second instructions and a plurality of prompts are associated with each other, acquires an evaluation value obtained by evaluating the plurality of prompts identified by the acquired degree of conformity, and displays one or a plurality of prompts to which the X-ray image is added selected from the plurality of prompts based on the evaluation value.

### CROSS-REFERENCE TO RELATED APPLICATIONS

This application is based upon and claims the benefit of priority from Japanese Patent Application No. 2024-156789, filed on Sep. 10, 2024; the entire contents of which are incorporated herein by reference.

### FIELD

Embodiments described herein relate generally to an X-ray image diagnostic apparatus, a medical information processing apparatus, a medical information processing system, and a medical information processing method.

### BACKGROUND

A user needs to input an appropriate prompt to obtain desired output from a generative model, such as large language models (LLMs). The skill to create a prompt, however, varies with users. As a result, the user may possibly fail to obtain the optimum output from the generative model. To address this, widely known are techniques to acquire a prompt corresponding to the user's question based on a database in which character strings and prompts are associated with each other.

The prompts in such a technique, however, are not based on evaluations by third parties, such as the user. Therefore, the prompts in the conventional technique may possibly fail to cause the generative model to generate an answer desired by the user.

### BRIEF DESCRIPTION OF THE DRAWINGS

FIG. 1 is a diagram of an example of a medical information processing system according to a first embodiment;

FIG. 2 is a diagram of an example of the data structure of a prompt database;

FIG. 3 is a diagram of an example of a prompt presentation image;

FIG. 4 is a flowchart of an example of a generation process performed by a medical information processing apparatus according to the first embodiment;

FIG. 5 is a diagram of an example of the medical information processing system according to a second embodiment;

FIG. 6 is a flowchart of an example of the generation process performed by the medical information processing apparatus according to the second embodiment;

FIG. 7 is a diagram of an example of the medical information processing system according to a third embodiment;

FIG. 8 is a diagram of an example of the data structure of the prompt database;

FIG. 9 is a diagram of an example of the prompt presentation image;

FIG. 10 is a diagram of an example of the data structure of the prompt database;

FIG. 11 is a diagram of an example of the prompt presentation image;

FIG. 12 is a diagram of an example of the data structure of the prompt database;

FIG. 13 is a diagram of an example of the prompt presentation image;

FIG. 14 is a diagram of an example of the prompt presentation image; and FIG. 15 is a diagram of an example of the medical information processing system according to a fourth embodiment.

### DETAILED DESCRIPTION

An X-ray image diagnostic apparatus or a medical information processing apparatus according to an embodiment includes an X-ray irradiator that outputs an X-ray and an X-ray detector that detects the X-ray output by the X-ray irradiator and transmitted through a subject. The X-ray image diagnostic apparatus or the medical information processing apparatus that generates an X-ray image based on detection data obtained by detection by the X-ray detector includes processing circuitry. The processing circuitry receives a first instruction, receive an instruction to add the X-ray image, acquires the degree of conformity between the first instruction and a second instruction based on a database in which a plurality of the second instructions and a plurality of prompts are associated with each other, acquires an evaluation value obtained by evaluating the plurality of prompts identified by the acquired degree of conformity, and displays one or a plurality of prompts to which the X-ray image is added selected from the plurality of prompts based on the evaluation value.

An X-ray image diagnostic apparatus, a medical information processing apparatus, a medical information processing system, and a medical information processing method according to the present embodiment are described below with reference to the accompanying drawings. In the following embodiments, parts with like reference numerals are assumed to operate in the same manner, and duplicate explanations thereof are omitted as appropriate.

### First Embodiment

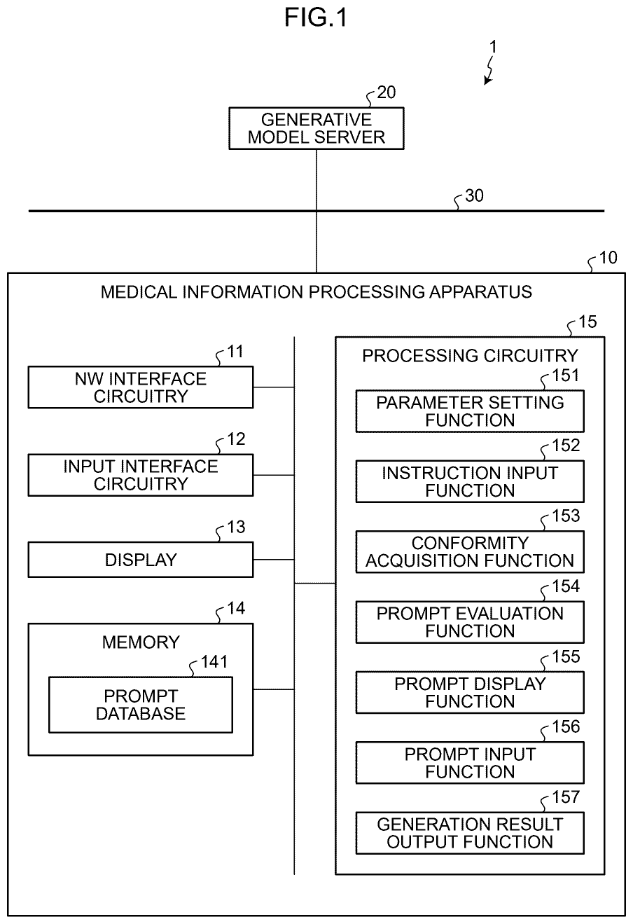

FIG. 1 is a diagram of an example of a medical information processing system 1 according to a first embodiment. The medical information processing system 1 includes a medical information processing apparatus 10 and a generative model server 20. The medical information processing apparatus 10 and the generative model server 20 are communicatively connected via a network 30. While the medical information processing system 1 illustrated in FIG. 1 includes one medical information processing apparatus 10 and one generative model server 20, the number of these apparatuses may be optionally changed. Furthermore, the medical information processing system 1 may include apparatuses and systems not illustrated in FIG. 1.

The medical information processing apparatus 10 is implemented by computer equipment, such as a personal computer and a workstation. The medical information processing apparatus 10 generates a prompt to be input to a generative model based on an input instruction input by a user. The medical information processing apparatus 10 transmits the generated prompt to the generative model server 20. The medical information processing apparatus 10 displays generation results output from the generative model server 20.

The generative model server 20 is implemented by computer equipment, such as a server and a workstation. The generative model server 20 inputs the prompt received from the medical information processing apparatus 10 to the generative model. Examples of the generative model include, but are not limited to, large language models (LLMs), large multimodal models (LMMs), etc. The generative model server 20 transmits the generation results output from the generative model to the medical information processing apparatus 10.

Next, an example of the medical information processing apparatus 10 is described in detail.

The medical information processing apparatus 10 includes network (NW) interface circuitry 11, input interface circuitry 12, a display 13, a memory 14, and processing circuitry 15.

The NW interface circuitry 11 is connected to the processing circuitry 15 and controls transmission and communications of various data to and from each apparatus connected via the network 30. The NW interface circuitry 11 is implemented by, for example, a network card, a network adapter, or a network interface controller (NIC).

The input interface circuitry 12 is connected to the processing circuitry 15, converts an input operation received from the user (medical worker) into electrical signals, and outputs them to the processing circuitry 15. Specifically, the input interface circuitry 12 converts an input operation received from the user into electrical signals and outputs them to the processing circuitry 15. The input interface circuitry 12 is implemented by, for example, a trackball, a switch button, a mouse, a keyboard, a touchpad on which the user performs an input operation by touching an operation surface, a touchscreen that integrates a display screen and a touchpad, a non-contact input circuit with an optical sensor, or a voice input circuit. In the present specification, the input interface circuitry 12 is not limited only to those with physical operation components, such as a mouse and a keyboard. Examples of the input interface circuitry 12 also include a processing circuit for electrical signals that receives electrical signals corresponding to an input operation from an external input device provided separately from the device and outputs the electrical signals to a control circuit.

The display 13 is connected to the processing circuitry 15 and displays various kinds of information and image data output from the processing circuitry 15. The display 13 is implemented by, for example, a liquid crystal display, a cathode ray tube (CRT) display, an organic EL display, a plasma display, or a touch panel.

The memory 14 is connected to the processing circuitry 15 and stores therein various data. The memory 14 stores therein various computer programs read and executed by the processing circuitry 15 to implement various functions. The memory 14 is implemented by, for example, a semiconductor memory element, such as a random access memory (RAM) and a flash memory, a hard disk, or an optical disc.

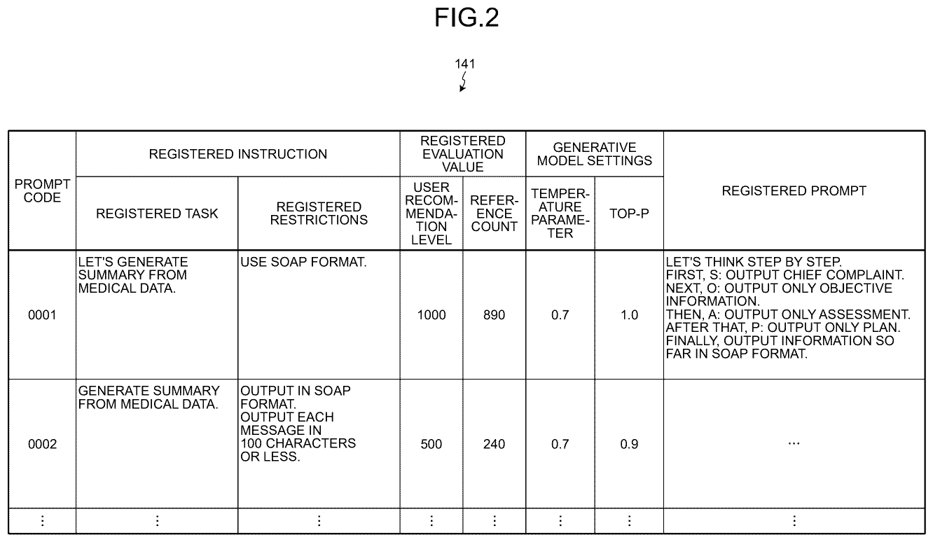

The memory 14 stores therein a prompt database 141, for example. FIG. 2 is a diagram of an example of the data structure of the prompt database 141. The prompt database 141 is a database storing therein information on prompts to be generated based on the instruction input by the user. The prompt database 141 stores therein a prompt code, a registered instruction, a registered evaluation value, generative model settings, and a registered prompt in a manner associated with one another.

The prompt code is identification information for identifying a prompt. The registered instruction is information indicating the instruction contents to be compared with the instruction input by the user. The registered instruction is text information, for example. The registered instruction includes a registered task and registered restrictions. The registered task is information indicating the instruction contents to be generated by the generative model. The registered restrictions are information indicating the restrictions on the instruction contents to be generated by the generative model.

The registered evaluation value is a value indicating the evaluation for the registered prompt. The registered evaluation value includes a user recommendation level and a reference count. The user recommendation level is information indicating the number of times the registered prompt is evaluated as a good prompt by users who use it.

The reference count is the number of times the registered prompt is displayed. The reference count may be the number of times the registered prompt is used. The registered evaluation value is not limited to the user recommendation level or the reference count and may include values obtained by evaluating the registered prompt by other methods. For example, the registered evaluation value may be a label indicating whether the registered prompt is a good prompt or may be a discrete value obtained by evaluating the registered prompt by the user on a plurality of grades, such as five grades.

The generative model settings are the settings for the generative model to input the registered prompt thereto. The generative model settings include a temperature parameter and TOP_P, for example. The temperature parameter and TOP_P are parameters that control the randomness of the generation results of the generative model. More random generation results are generated as the parameter value of the temperature parameter increases. More specifically, the temperature parameter is a parameter that controls the range in which a word is an object to be selected. More random generation results are generated as the parameter value of TOP_P increases. More specifically, TOP_P is a parameter that controls the probability distribution of whether a word is applicable.

The registered prompt is a prompt to be suggested to the user. In other words, the registered prompt is a prompt output when the input instruction conforming to the registered instruction is input. The registered prompt may be a set of prompts. If the registered prompt is a set of prompts, the user inputs one prompt to the generative model and then inputs the next prompt after they acquires the generation results from the generative model. Thus, the user may repeatedly perform the processing on the set of prompts.

The prompt database 141 may be a database of prompts that have previously been input to the generative model. In this case, the prompt database 141 may include the generation results generated by inputting the prompts to the generative model.

The processing circuitry 15 controls the entire operations of the medical information processing apparatus 10. The processing circuitry 15 includes a parameter setting function 151, an instruction input function 152, a conformity acquisition function 153, a prompt evaluation function 154, a prompt display function 155, a prompt input function 156, and a generation result output function 157, for example. In the embodiment, processing functions implemented by the parameter setting function 151, the instruction input function 152, the conformity acquisition function 153, the prompt evaluation function 154, the prompt display function 155, the prompt input function 156, and the generation result output function 157 serving as the components are stored in the memory 14 as computer programs executable by a computer. The processing circuitry 15 is a processor that reads each computer program from the memory 14 and executes it to implement the function corresponding to the computer program. In other words, the processing circuitry 15 that reads the computer programs includes the functions in the processing circuitry 15 illustrated in FIG. 1.

In FIG. 1, a single processor implements the parameter setting function 151, the instruction input function 152, the conformity acquisition function 153, the prompt evaluation function 154, the prompt display function 155, the prompt input function 156, and the generation result output function 157. Alternatively, a plurality of independent processors may be combined and constitute the processing circuitry 15, and each processor may execute the computer program to implement the function. In FIG. 1, a single storage circuit, such as the memory 14, stores therein the computer programs corresponding to the processing functions. Alternatively, a plurality of storage circuits may be provided in a distributed manner, and the processing circuitry 15 may read the corresponding computer program from the individual storage circuit.

The term “processor” used in the description above refers to, for example, a central processing unit (CPU), a graphical processing unit (GPU), or a circuit, such as an application specific integrated circuit (ASIC) and a programmable logic device (e.g., a simple programmable logic device (SPLD), a complex programmable logic device (CPLD), and a field programmable gate array (FPGA)). The processor reads and executes the computer programs stored in the memory 14 to implement the functions. Instead of being stored in the memory 14, the computer programs may be incorporated directly in the circuit of the processor. In this case, the processor reads and executes the computer programs incorporated in the circuit to implement the functions.

The parameter setting function 151 sets parameters relating to the generative model. For example, the parameter setting function 151 sets the temperature parameter and TOP_P in the prompt database 141. The parameter setting function 151 does not necessarily set these parameters and may set other parameters.

The instruction input function 152 receives an input instruction for the generative model from the user. The instruction input function 152 is an example of an input unit. The input instruction is an example of a first instruction. The input instruction is information indicating the instruction contents for the generative model input by the user. The instruction input function 152 may receive the input instruction in a text form via the input interface circuitry 12, receive the input instruction by voice, or receive the input instruction in other forms.

The input instruction includes an input task desired to be generated by the generative model and input restrictions on the generation results of the generative model. The input task is information indicating the contents of the task desired to be performed by the generative model. The input task is, for example, information indicating the generation contents desired to be generated by the generative model. For example, the input task is information, such as “generate a summary from the medical data”.

The input restrictions are information indicating the restrictions on generating the information on the generation contents indicated by the input task. The input restrictions are information, such as an output format and preconditions. For example, the input restrictions are information, such as “output in the SOAP format”. The SOAP format is a format for describing information in medical records and in which the course of medical treatment is described in the order of subject (subjective information), object (objective information), assessment (evaluation), and plan (plan).

Furthermore, the instruction input function 152 may receive the input instruction including medical information. In the instruction input function 152, for example, medical information may be included in the input task, like “generate a summary from the medical data”. In the instruction input function 152, a restriction on medical information may be included in the input restriction, like “output in the SOAP format”.

The conformity acquisition function 153 acquires the degree of conformity between the input instruction and the registered instruction based on the prompt database 141 in which a plurality of registered instructions for the generative model and a plurality of registered prompts are associated with each other. The conformity acquisition function 153 is an example of an acquisition unit. The registered instruction is an example of a second instruction. In other words, the conformity acquisition function 153 acquires the degree of conformity between each registered instruction stored in the prompt database 141 and the input instruction input by the instruction input function 152. The degree of conformity is information indicating the degree to which the input instruction conforms to the registered instruction stored in the prompt database 141.

The conformity acquisition function 153 acquires the degree of conformity based on rules set in advance, for example. Specifically, the conformity acquisition function 153 acquires the degree of conformity by the number of times of matching in pattern matching by regular expression and the degree of matching of character strings until a new line is started.

The conformity acquisition function 153 may acquire the degree of conformity based on a learned model generated by machine learning. Specifically, the conformity acquisition function 153 acquires the degree of conformity using the results of determining whether a specific keyword included in the input instruction can be extracted from the registered instruction or the results of determining whether a specific definition included in the input instruction can be extracted from the registered instruction based on the learned model.

The conformity acquisition function 153 may acquire the degree of conformity based on the similarity of character strings or words between the input instruction and the registered instruction. Specifically, the conformity acquisition function 153 acquires the degree of conformity based on the edit distance. Alternatively, the conformity acquisition function 153 acquires the degree of conformity based on the cosine similarity or the word mover's distance between feature vectors, such as Bow, TF-IDF, and machine learning encoder output.

The conformity acquisition function 153 may acquire the degree of conformity based on the matching ratio of words between the input instruction and the registered instruction. Specifically, the conformity acquisition function 153 acquires the degree of conformity based on the matching ratio of named entity or the matching ratio by Gestalt pattern matching.

The conformity acquisition function 153 may acquire the degree of conformity based on an operation by the user.

The conformity acquisition function 153 may acquire the degree of conformity not only by comparing the input instruction with the registered instruction but also considering other factors. The conformity acquisition function 153 may acquire the degree of conformity based on the parameters of the generative model. For example, the conformity acquisition function 153 may acquire the degree of conformity based on the generative model settings in the prompt database 141 and the settings of the temperature parameter and TOP_P in the generative model server 20.

The prompt evaluation function 154 acquires an evaluation value obtained by evaluating a plurality of registered prompts identified by the degree of conformity acquired by the conformity acquisition function 153. The prompt evaluation function 154 is an example of a first evaluation unit. More specifically, the prompt evaluation function 154 evaluates the registered prompts the degree of conformity of which is equal to or higher than a threshold based on the registered evaluation value stored in the prompt database 141. In other words, the prompt evaluation function 154 acquires the registered evaluation value including the user recommendation level or the reference count. The user recommendation level is information indicating the number of times the registered prompt is displayed as a good prompt by the user. The reference count is information indicating the number of times the prompt is evaluated. For example, the prompt evaluation function 154 evaluates whether the registered prompt corresponding to the registered instruction determined to conform to the input instruction based on the degree of conformity is a prompt suitable for the input instruction.

The prompt display function 155 displays one or a plurality of prompts selected from the plurality of prompts based on the results of evaluation by the prompt evaluation function 154. The prompt display function 155 is an example of a display control unit. In other words, the prompt display function 155 displays the registered prompt suitable for the input instruction based on the results of evaluation by the prompt evaluation function 154. For example, the prompt display function 155 displays a prompt presentation image G1 that presents the registered prompt suitable for the input instruction on the display 13.

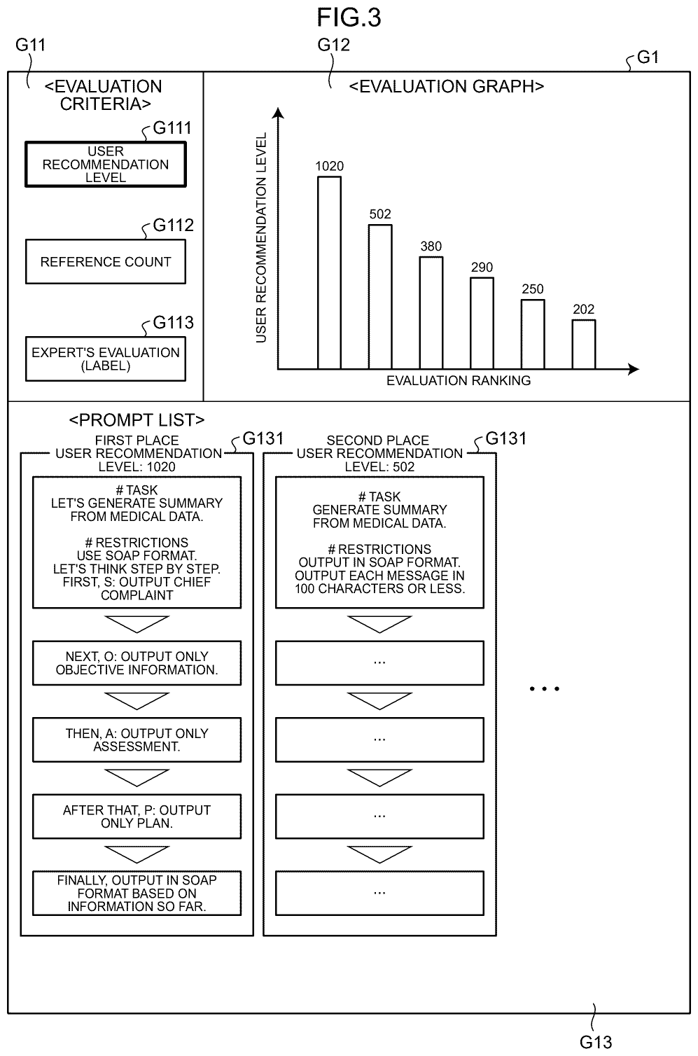

FIG. 3 is a diagram of an example of the prompt presentation image G1. The prompt presentation image G1 includes an evaluation criteria area G11, an evaluation graph area G12, and a prompt list area G13.

The evaluation criteria area G11 is an area that displays buttons for specifying the evaluation criterion for the registered prompts stored in the prompt database 141. The evaluation criteria area G11 includes a user recommendation level button G111, a reference count button G112, and an expert's evaluation button G113, for example. The user recommendation level button G111 is a button that receives an operation to specify the user recommendation level as the evaluation criterion. The reference count button G112 is a button that receives an operation to specify the reference count as the evaluation criterion. The expert's evaluation button G113 is a button that receives an operation to specify the expert's evaluation as the evaluation criterion. The prompt evaluation function 154 evaluates the registered prompt based on the evaluation criterion specified in the evaluation criteria area G11.

The evaluation graph area G12 is an area that displays a bar graph indicating the evaluation value obtained by evaluation by the prompt evaluation function 154. The prompt evaluation function 154 evaluates each registered prompt based on the evaluation criterion specified in the evaluation criteria area G11. The evaluation graph area G12 is an area that displays a bar graph indicating the evaluation value of each registered prompt evaluated by the prompt evaluation function 154. The evaluation graph area G12 illustrated in FIG. 3 indicates a bar graph of evaluation according to the user recommendation level.

The prompt list area G13 is an area where prompt images G131 are arranged in the order of the evaluation value obtained by evaluation by the prompt evaluation function 154.

The prompt image G131 is an image including an evaluation ranking of the registered prompt, the evaluation value of the registered prompt, the input instruction, and the registered prompt. More specifically, the prompt display function 155 displays the registered prompt that requests the processing of medical information according to the instruction contents indicated by the input instruction. Thus, the registered prompts are arranged in the order of the evaluation value, thereby enabling the user to select the prompt suitable for the input instruction.

The prompt input function 156 receives an operation to select a prompt to be input to the generative model from the registered prompts arranged in the prompt list area G13. The prompt input function 156 transmits the registered prompt selected from the prompt list area G13 to the generative model server 20 as the prompt to be input to the generative model. In other words, the prompt input function 156 transmits the registered prompt selected from one or more registered prompts displayed by the prompt display function 155 to the generative model server 20. The prompt input function 156 is an example of a transmission unit.

The prompt input function 156 may receive an operation to select the prompt to be input to the generative model by any method. For example, the prompt input function 156 may receive an operation of copying the registered prompt in the prompt image G131 and pasting it into a prompt input field for the generative model.

When receiving an operation to select the registered prompt in the prompt image G131, the prompt input function 156 may input the registered prompts serving as a set of prompts in the order of being presented, for example. In other words, when the registered prompt is selected, the prompt input function 156 inputs the first registered prompt to the generative model. When the generation results corresponding to the first registered prompt are output from the generative model, the prompt input function 156 inputs the second registered prompt to the generative model. When the generation results corresponding to the second registered prompt are output from the generative model, the prompt input function 156 inputs the third registered prompt to the generative model. The prompt input function 156 may repeatedly perform the processing described above until it inputs the last registered prompt to the generative model.

The generative model server 20 causes the generative model to perform the processing based on the prompt transmitted by the prompt input function 156. In other words, the generative model server 20 performs the processing by inputting the prompt to the generative model. The generative model server 20 transmits the generation results generated by the generative model to the medical information processing apparatus 10.

The generation result output function 157 outputs the generation results of the generative model corresponding to the prompt input by the prompt input function 156. More specifically, the generation result output function 157 receives the generation results of the generative model from the generative model server 20. The generation result output function 157 displays the generation results of the generative model corresponding to the prompt on the display 13.

Next, the generation process performed by the medical information processing apparatus 10 is described.

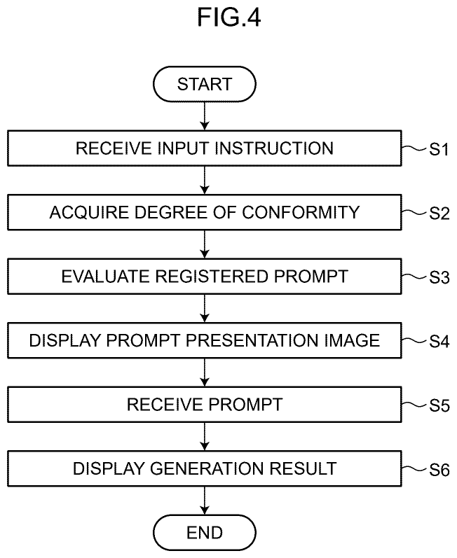

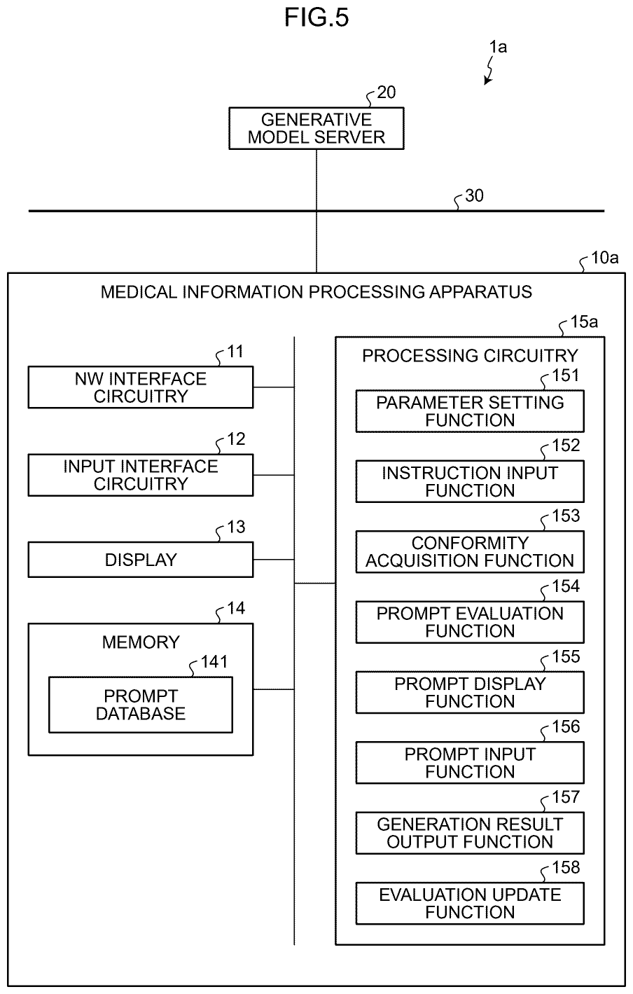

FIG. 4 is a flowchart of an example of the generation process performed by the medical information processing apparatus 10 according to the first embodiment.

The instruction input function 152 receives an input instruction indicating the instruction contents for the generative model (Step S1).

The conformity acquisition function 153 acquires the degree of conformity between the registered instruction stored in the prompt database 141 and the input instruction input by the instruction input function 152 (Step S2).

The prompt evaluation function 154 evaluates one or a plurality of registered prompts identified by the degree of conformity acquired by the conformity acquisition function 153 (Step S3).

The prompt display function 155 displays the prompt presentation image G1 that presents the registered prompt suitable for the input instruction based on the results of evaluation of the registered prompt on the display 13 (Step S4).

The prompt input function 156 inputs the registered prompt selected in the prompt presentation image G1 to the generative model (Step S5). In other words, the prompt input function 156 transmits the selected registered prompt to the generative model server 20.

The generation result output function 157 displays the generation results generated by the generative model on the display 13 (Step S6).

After that, the medical information processing apparatus 10 terminates the generation process.

As described above, the medical information processing apparatus 10 according to the first embodiment receives an input instruction for the generative model. The medical information processing apparatus 10 acquires the degree of conformity between the input instruction and the registered instruction based on the prompt database 141. The medical information processing apparatus 10 displays the registered prompt identified by the registered evaluation value out of the registered prompts identified by the degree of conformity on the display 13. Thus, the medical information processing apparatus 10 displays the prompt identified by the registered evaluation value. Therefore, the medical information processing apparatus 10 can output the prompt that causes the generative model to generate an answer desired by the user.

As described above, when the processing circuitry 15 of the medical information processing apparatus 10 receives an input instruction for the generative model, it accesses the memory 14 that stores therein the prompt database 141 having a plurality of prompts. The processing circuitry 15 of the medical information processing apparatus 10 displays one or a plurality of prompts selected from the plurality of prompts stored in the prompt database 141 on the display 13. With this configuration, the processing circuitry 15 of the medical information processing apparatus 10 can display the prompt suitable for the input instruction with fewer hardware resources than when it generates the prompt.

The processing circuitry 15 of the medical information processing apparatus 10 transmits the prompt to the generative model server 20 including the generative model and receives the generation results generated by the generative model by inputting the prompt from the generative model server 20. The processing circuitry 15 of the medical information processing apparatus 10 displays the received generation results on the display 13. Thus, the medical information processing system 1 enables a plurality of medical information processing apparatuses 10 to share the generative model, whereby each of the medical information processing apparatuses 10 need not store therein the generative model. Therefore, the medical information processing system 1 can save the memory capacity of each of the medical information processing apparatuses 10.

### Second Embodiment

A medical information processing apparatus 10a according to a second embodiment includes an evaluation update function 158 that updates the registered evaluation value stored in the prompt database 141.

FIG. 5 is a diagram of an example of a medical information processing system 1a according to the second embodiment. The medical information processing system 1a includes the medical information processing apparatus 10a. processing circuitry 15a of the medical information processing apparatus 10a includes the parameter setting function 151, the instruction input function 152, the conformity acquisition function 153, the prompt evaluation function 154, the prompt display function 155, the prompt input function 156, the generation result output function 157, and the evaluation update function 158.

The parameter setting function 151, the instruction input function 152, the conformity acquisition function 153, the prompt evaluation function 154, the prompt display function 155, the prompt input function 156, and the generation result output function 157 have the same functions as those according to the first embodiment.

The evaluation update function 158 updates the registered evaluation value stored in the prompt database 141. The evaluation update function 158 is an example of an update unit. More specifically, when the generation results of the generative model are output by the generation result output function 157, the evaluation update function 158 displays an evaluation input image for inputting the evaluation of the prompt input to the generative model that generates the output generation results. In other words, the evaluation input image is an image that receives the user's evaluation of the registered prompt serving as the prompt input to the generative model. More specifically, the evaluation input image receives an evaluation of whether the registered prompt is a prompt that generates the generation results intended by the user.

For example, the evaluation input image may be an image that receives an evaluation of whether the prompt is good or bad, such as a good button and a bad button. Alternatively, the evaluation input image may be an image that receives an operation to select a grade corresponding to the user's evaluation in five-grade evaluation. Still alternatively, the evaluation input image may be an image that receives an operation to specify a score corresponding to the user's evaluation.

The evaluation update function 158 updates one item of the registered evaluation value based on the evaluation results received in the evaluation input image. The evaluation update function 158 does not necessarily update one item of the registered evaluation value and may update a plurality of items.

The evaluation update function 158 updates the registered evaluation value at any desired timing. For example, when a set time comes, the evaluation update function 158 may update the registered evaluation value in the prompt database 141 to reflect the evaluation results up to the set time. In other words, the evaluation update function 158 may update the registered evaluation value in the prompt database 141 to reflect the evaluation results of the previous day at 24 o'clock, for example.

Alternatively, the evaluation update function 158 may update the registered evaluation value in the prompt database 141 to reflect the evaluation results of the previous day when receiving an explicit operation on an update button or the like.

The prompt evaluation function 154 evaluates one or a plurality of registered prompts identified by the degree of conformity acquired by the conformity acquisition function 153 based on the registered evaluation value in the prompt database 141 updated by the evaluation update function 158.

Next, the generation process performed by the medical information processing apparatus 10a is described.

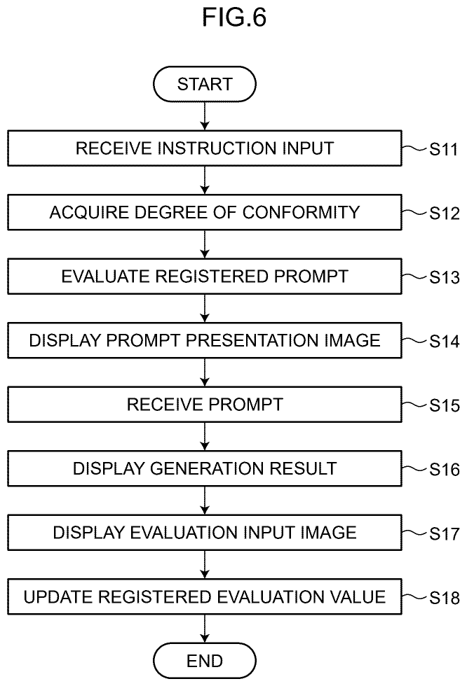

FIG. 6 is a flowchart of an example of the generation process performed by the medical information processing apparatus 10a according to the second embodiment.

The instruction input function 152 receives an input instruction indicating the instruction contents for the generative model (Step S11).

The conformity acquisition function 153 acquires the degree of conformity between the registered instruction stored in the prompt database 141 and the input instruction input by the instruction input function 152 (Step S12).

The prompt evaluation function 154 evaluates one or a plurality of registered prompts identified by the degree of conformity acquired by the conformity acquisition function 153 (Step S13).

The prompt display function 155 displays the prompt presentation image G1 that presents the registered prompt suitable for the input instruction based on the results of evaluation of the registered prompt on the display 13 (Step S14).

The prompt input function 156 inputs the registered prompt selected in the prompt presentation image G1 to the generative model (Step S15). In other words, the prompt input function 156 transmits the selected registered prompt to the generative model server 20.

The generation result output function 157 displays the generation results generated by the generative model on the display 13 (Step S16). In other words, the generation result output function 157 receives the generation results from the generative model server 20.

The evaluation update function 158 displays the evaluation input image that receives an evaluation of whether the registered prompt is a prompt that causes the generative model to generate the generation results intended by the user on the display 13 (Step S17). In other words, the evaluation update function 158 receives an evaluation of whether the registered prompt is a prompt that causes the generative model to generate the generation results intended by the user.

When the update conditions for updating the registered evaluation value in the prompt database 141 are satisfied, the evaluation update function 158 updates the registered evaluation value so as to reflect the evaluation results obtained until the update conditions are satisfied (Step S18).

After that, the medical information processing apparatus 10a terminates the generation process.

As described above, the medical information processing apparatus 10a according to the second embodiment includes the evaluation update function 158 that updates the registered evaluation value in the prompt database 141. The evaluation update function 158 can update the registered evaluation value, such as the user recommendation level, as appropriate. Therefore, the medical information processing apparatus 10a can output the prompt that causes the generative model to generate an answer desired by the user that changes over time.

### Third Embodiment

A medical information processing apparatus 10b according to a third embodiment evaluates the registered prompt also based on the reproducibility of the generation results generated by the generative model.

When the same prompt is input at different times, the generative model may generate different generation results depending on the times. For example, the generative model may generate different generation results when the version is changed or when machine learning, such as additional learning and federated learning, is performed on the generative model. As a result, the user may possibly fail to acquire the expected generation results if they input a registered prompt proven in the past to the generative model. To address this, the medical information processing apparatus 10b evaluates the registered prompts also based on the reproducibility of the generation results generated by the generative model.

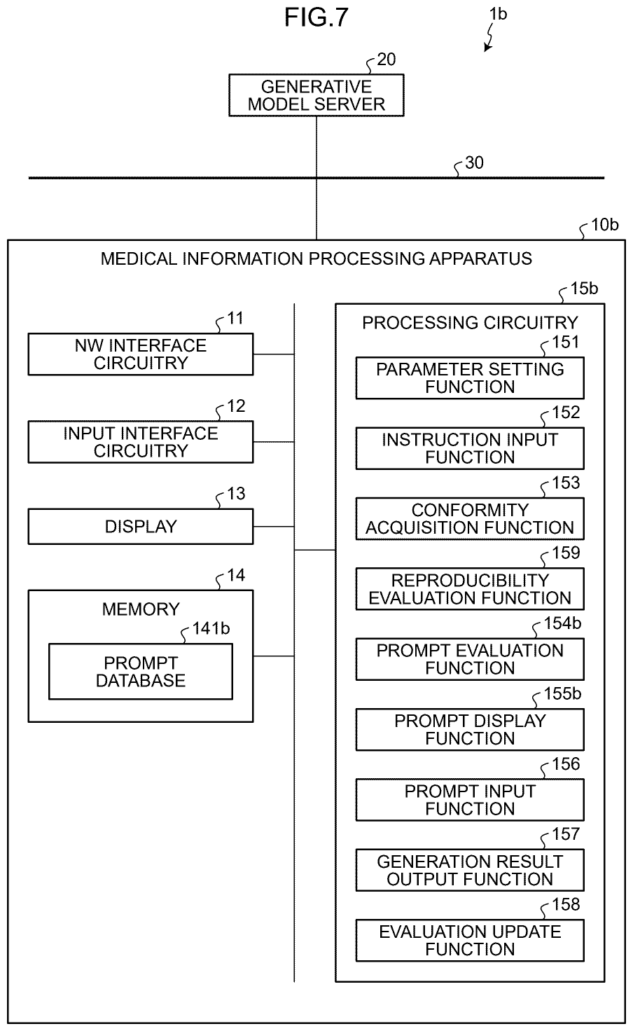

FIG. 7 is a diagram of an example of a medical information processing system 1b according to the third embodiment. The medical information processing system 1b includes the medical information processing apparatus 10b.

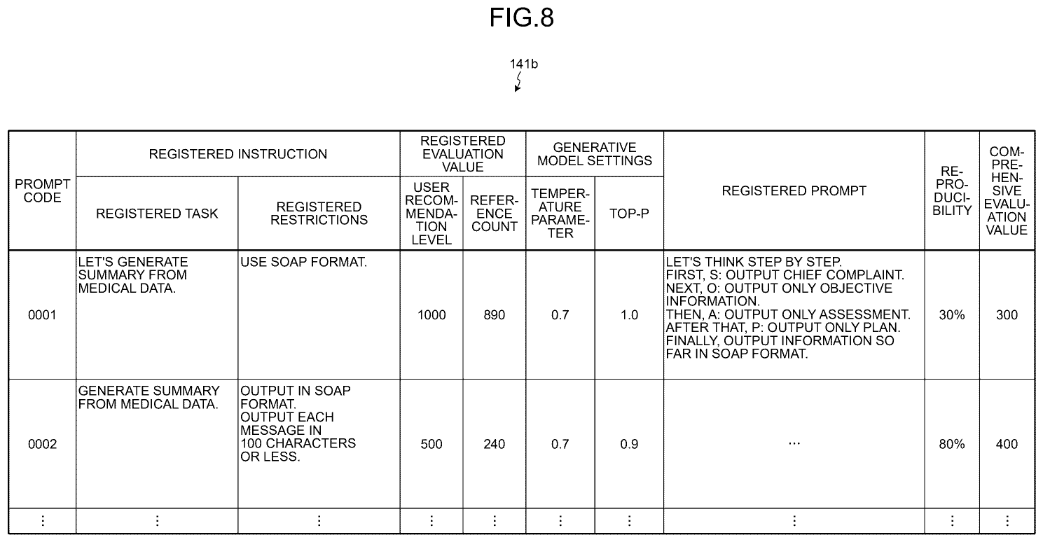

The memory 14 of the medical information processing apparatus 10b stores therein a prompt database 141b. FIG. 8 is a diagram of an example of the data structure of the prompt database 141b. The prompt database 141b stores therein the prompt code, the registered instruction, the registered evaluation value, the generative model settings, the registered prompt, reproducibility, and a comprehensive evaluation value in a manner associated with one another.

The prompt code, the registered instruction, the registered evaluation value, the generative model settings, and the registered prompt are the same information as those according to the first embodiment.

The reproducibility is information indicating the degree of identicalness between the generation results generated by the generative model by inputting the registered prompt and past generation results. In other words, the reproducibility is information indicating the degree of identicalness between the past generation results of the generative model and the generation results of the present generative model. The reproducibility may indicate the degree of identicalness between the generation results of the generative model obtained when the registered prompt is created and those of the present generative model, the degree of identicalness between the generation results of the previous generative model and those of the present generative model, or comparison between the generation results generated at other timings.

The past generation results of the generative model may be included in the prompt database 141b or stored in other storage media.

The comprehensive evaluation value is a value based on the registered evaluation value and the reproducibility. The comprehensive evaluation value is, for example, a value obtained by multiplying the user recommendation level of the registered evaluation value by the reproducibility. The comprehensive evaluation value is not limited to a value obtained by multiplying the user recommendation level by the reproducibility. It may be a value obtained by multiplying the reference count by the reproducibility, a value obtained by multiplying each value of the registered evaluation value resulting from an arithmetic operation by the reproducibility, or a value obtained by multiplying other values by the reproducibility.

processing circuitry 15b of the medical information processing apparatus 10b includes the parameter setting function 151, the instruction input function 152, the conformity acquisition function 153, a reproducibility evaluation function 159, a prompt evaluation function 154b, a prompt display function 155b, the prompt input function 156, the generation result output function 157, and the evaluation update function 158.

The parameter setting function 151, the instruction input function 152, the conformity acquisition function 153, the prompt input function 156, the generation result output function 157, and the evaluation update function 158 have the same functions as those according to the second embodiment. The processing circuitry 15b does not necessarily include the evaluation update function 158.

The reproducibility evaluation function 159 evaluates the reproducibility indicating the degree of identicalness between the generation results generated by the generative model by inputting the registered prompt and the past generation results. The reproducibility evaluation function 159 is an example of an evaluation unit. In other words, the reproducibility evaluation function 159 evaluates the reproducibility of the registered prompt based on the past generation results of the generative model and the present generation results of the generative model.

For example, the reproducibility evaluation function 159 acquires the reproducibility based on the similarity of character strings or words included in the generation results. Specifically, the reproducibility evaluation function 159 acquires the reproducibility based on the edit distance. Alternatively, the reproducibility evaluation function 159 acquires the reproducibility based on the cosine similarity or the word mover's distance between feature vectors, such as Bow, TF-IDF, and machine learning encoder output.

The reproducibility evaluation function 159 acquires the reproducibility based on the matching ratio of words. Specifically, the reproducibility evaluation function 159 acquires the reproducibility based on the matching ratio of named entity or the matching ratio by Gestalt pattern matching.

The reproducibility evaluation function 159 registers the acquired reproducibility in the prompt database 141b.

The prompt evaluation function 154b acquires the comprehensive evaluation value by multiplying the acquired reproducibility by the user recommendation level in the prompt database 141b. The prompt evaluation function 154b registers the acquired comprehensive evaluation value in the prompt database 141b.

The prompt display function 155b displays a prompt presentation image G1b based on the comprehensive evaluation value according to the registered evaluation value and the reproducibility. This configuration enables the user to select the registered prompt while considering the reproducibility.

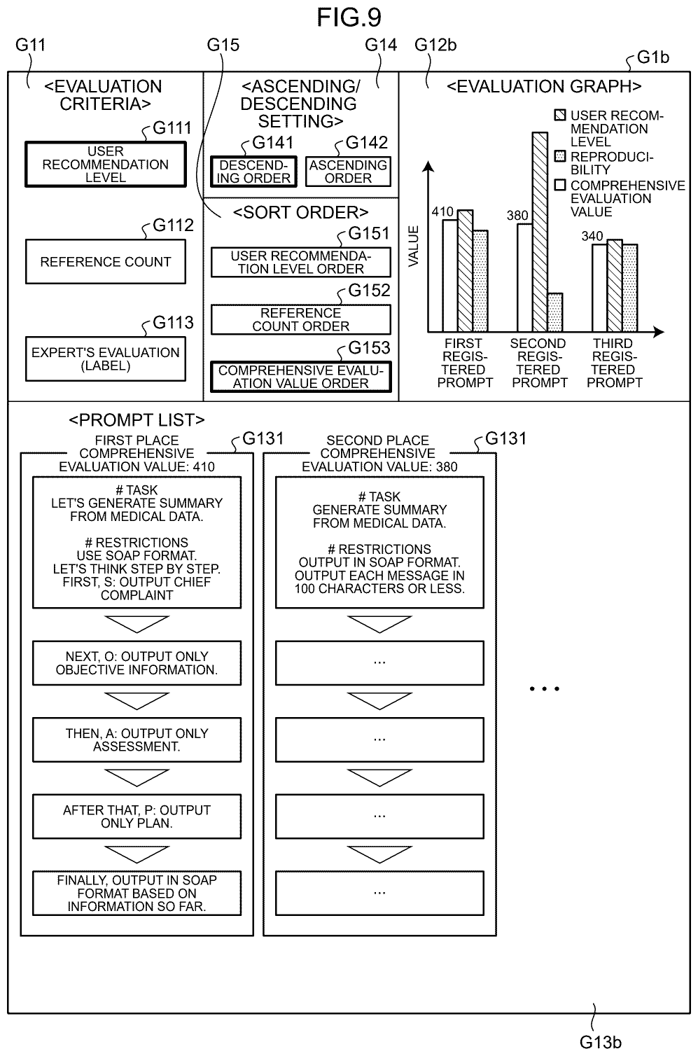

FIG. 9 is a diagram of an example of the prompt presentation image G1b. The prompt presentation image G1b includes the evaluation criteria area G11, an evaluation graph area G12b, a prompt list area G13b, an ascending/descending setting area G14, and a sort order area G15.

The evaluation reference area G11 is the same as the evaluation reference area G11 illustrated in FIG. 3.

The sort order area G15 is an area that receives an operation to specify the criterion for sorting the registered prompts in the evaluation graph area G12b and the prompt list area G13b. The sort order area G15 includes a user recommendation level order button G151, a reference count order button G152, and a comprehensive evaluation order button G153. The user recommendation level order button G151 is a button that receives specification to arrange the registered prompts in the order of the user recommendation level in the evaluation graph area G12b and the prompt list area G13b. The reference count order button G152 is a button that receives specification to arrange the registered prompts in the order of the reference count in the evaluation graph area G12b and the prompt list area G13b. The comprehensive evaluation order button G153 is a button that receives specification to arrange the registered prompts in the order of the comprehensive evaluation in the evaluation graph area G12b and the prompt list area G13b.

The ascending/descending setting area G14 is an area that receives an operation to specify the order of the sort selected in the sort order area G15 in the evaluation graph area G12b and the prompt list area G13b. The ascending/descending setting area G14 includes a descending order button G141 and an ascending order button G142. The descending order button G141 is a button that receives an operation to make a setting to arrange the registered prompts in descending order. The ascending order button G142 is a button that receives an operation to make a setting to arrange the registered prompts in ascending order.

The evaluation graph area G12b is an area that displays a bar graph indicating the registered evaluation value, the reproducibility, and the comprehensive evaluation value of each registered prompt stored in the prompt database 141b. The registered evaluation value is a value set in the evaluation criteria area G11 out of the evaluation values obtained by evaluation by the prompt evaluation function 154b. The reproducibility is a value indicating the reproducibility acquired by the reproducibility evaluation function 159. The comprehensive evaluation value is a value acquired by the prompt evaluation function 154b. In the evaluation graph area G12b, the registered prompts stored in the prompt database 141b are arranged in the order specified in the ascending/descending setting area G14 based on the criterion specified in the sort order area G15.

The prompt list area G13b is an area where the prompt images G131 are arranged in the order specified in the ascending/descending setting area G14 based on the criterion specified in the sort order area G15.

As described above, the medical information processing apparatus 10b according to the third embodiment evaluates the registered prompts also based on the reproducibility between the past generation results of the generative model and the present generation results of the generative model. Therefore, the medical information processing apparatus 10b can prevent the prompts that generate unexpected generation results from being displayed.

### First Modification

When the reproducibility is lower than a threshold, the medical information processing apparatus 10b notifies the user of low reproducibility.

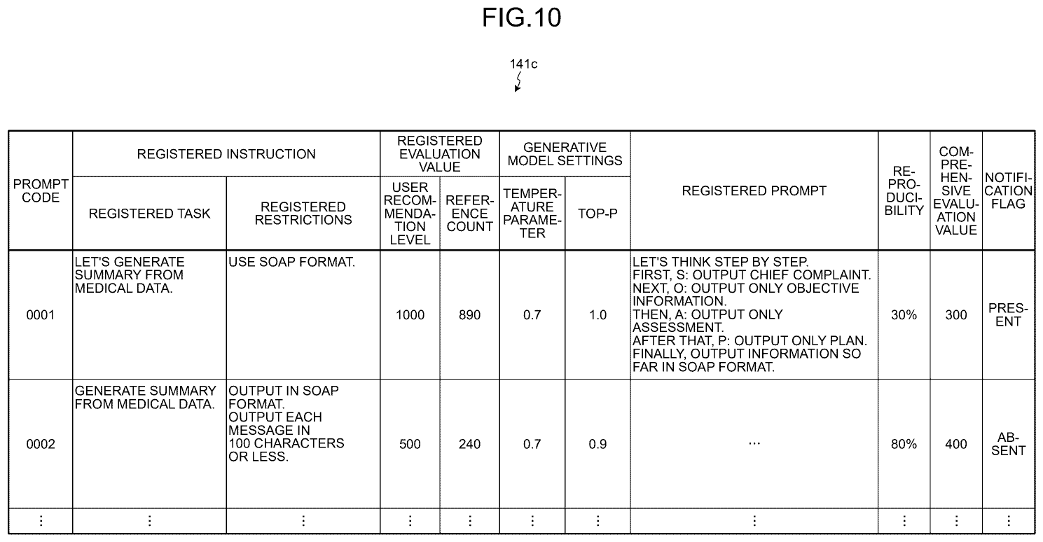

FIG. 10 is a diagram of an example of the data structure of a prompt database 141c. The prompt database 141c stores therein the prompt code, the registered instruction, the registered evaluation value, the generative model settings, the registered prompt, the reproducibility, the comprehensive evaluation value, and a notification flag in a manner associated with one another. The prompt code, the registered instruction, the registered evaluation value, the generative model settings, the registered prompt, the reproducibility, and the comprehensive evaluation value are the same information as those according to the first modification.

The notification flag is a flag indicating whether to notify the user of low reproducibility. The notification flag is set to “with notification” when the associated reproducibility is lower than the threshold. For example, the notification flag is set to “with notification” when the reproducibility is lower than 60%.

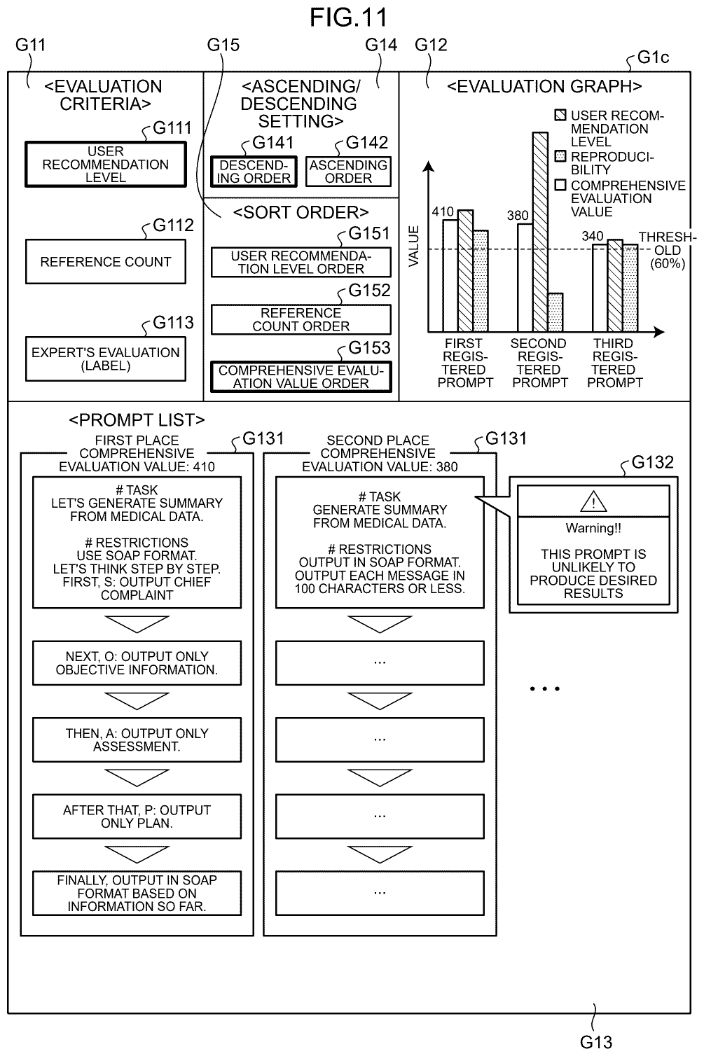

FIG. 11 is a diagram of an example of a prompt presentation image G1c. The prompt presentation image G1c includes the evaluation criteria area G11, the evaluation graph area G12, the prompt list area G13, the ascending/descending setting area G14, and the sort order area G15.

The prompt display function 155b displays a notification image G132 indicating that the reproducibility of the registered prompt is lower than the threshold. More specifically, the prompt display function 155b displays the notification image G132 in the prompt list area G13 when the notification flag corresponding to the registered prompt indicated by the prompt image G131 is set to “with notification”. In other words, the prompt display function 155b displays the notification image G132 in association with the corresponding prompt image G131.

The notification image G132 is an image that notifies the user that they fail to acquire the expected generation results from the generative model due to low reproducibility. For example, the notification image G132 includes the message “this prompt is unlikely to produce the desired results”.

### Second Modification

The medical information processing apparatus 10b does not present the registered prompt with a reproducibility lower than a threshold.

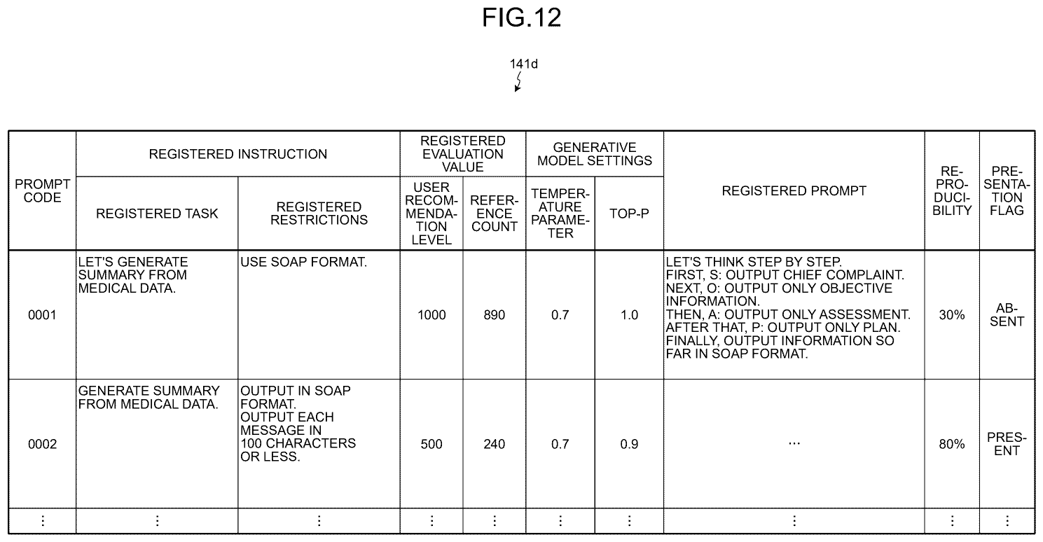

FIG. 12 is a diagram of an example of the data structure of a prompt database 141d. The prompt database 141d stores therein the prompt code, the registered instruction, the registered evaluation value, the generative model settings, the registered prompt, the reproducibility, the comprehensive evaluation value, and a presentation flag in a manner associated with one another. The prompt code, the registered instruction, the registered evaluation value, the generative model settings, the registered prompt, the reproducibility, and the comprehensive evaluation value are the same information as those according to the first modification.

The presentation flag is a flag indicating whether to present the associated registered prompt to the user. In other words, the presentation flag is a flag indicating whether to hide the registered prompt.

The reproducibility evaluation function 159 registers “with presentation” in the presentation flag of the prompt database 141d when the reproducibility is equal to or higher than the threshold. The reproducibility evaluation function 159 registers “without presentation” in the presentation flag of the prompt database 141d when the reproducibility is lower than the threshold. For example, the reproducibility evaluation function 159 registers “without presentation” in the presentation flag of the prompt database 141d when the reproducibility is lower than 60%.

The prompt display function 155b determines whether to display the registered prompt based on the presentation flag in the prompt database 141d. When “with presentation” is registered in the presentation flag, the prompt display function 155b displays the registered prompt in the prompt presentation image G1c based on the registered evaluation value and the comprehensive evaluation value in the prompt database 141d. When “without presentation” is registered in the presentation flag, the prompt display function 155b does not display the registered prompt in the prompt presentation image G1c regardless of the registered evaluation value or the comprehensive evaluation value. The prompt display function 155b does not display the registered prompt with a reproducibility lower than the threshold. With this configuration, the medical information processing apparatus 10b prevents unexpected generation results from being output from the generative model.

The prompt database 141d does not necessarily include the presentation flag and may include other flags. For example, the prompt database 141d may include a deletion flag that requests deletion of the associated registered prompt or a stop flag that requests update of the associated registered prompt.

### Third Modification

The medical information processing apparatuses 10, 10a, and 10b receive not only the input instruction in a text form but also the input instruction with additional information.

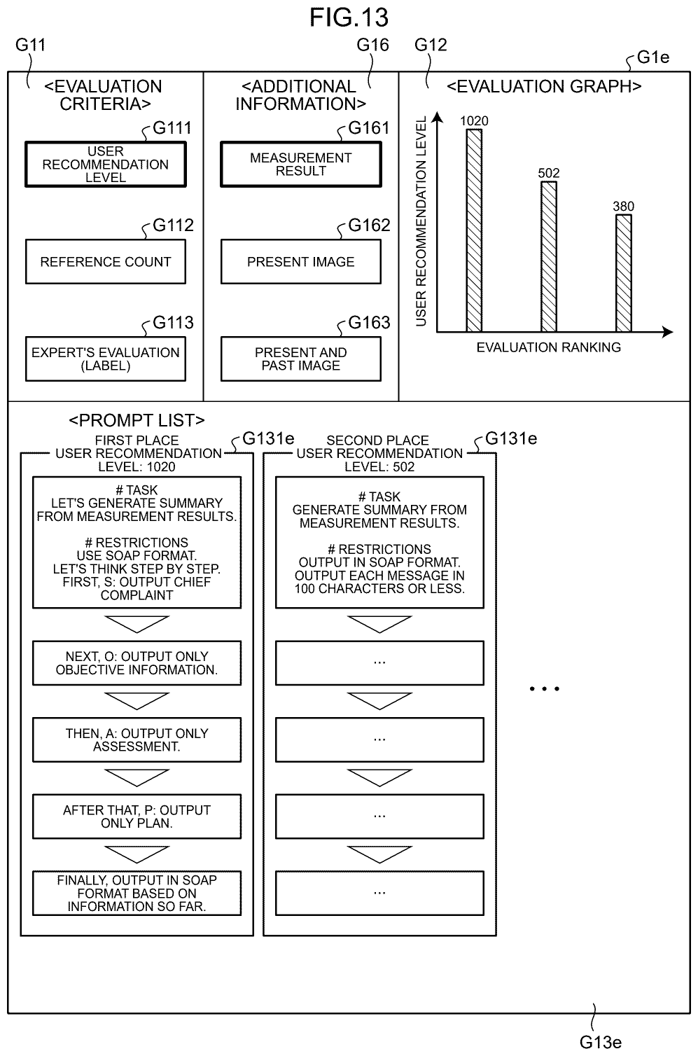

FIG. 13 is a diagram of an example of a prompt presentation image G1e. The prompt display functions G155 and G155b display the prompt presentation image G1e on the display 13. The prompt presentation image G1e includes the evaluation criteria area G11, the evaluation graph area G12, a prompt list area G13e, and an additional information area G16.

The evaluation criteria area G11 and the evaluation graph area G12 are the same as those in the prompt presentation image G1 illustrated in FIG. 3.

The additional information area G16 is an area that receives an operation to specify information to be added to the prompt to be input to the generative model. The additional information area G16 includes a measurement result button G161, a present image button G162, and a present and past image button G163. The measurement result button G161, the present image button G162, and the present and past image button G163 are examples of a specification unit.

The measurement result button G161 is a button that receives an operation to add measurement results to the prompt. When the measurement result button G161 is pressed, the prompt display functions G155 and G155b receive an operation to specify the measurement results to be added to the prompt.

The present image button G162 is a button that receives an operation to add a present image to the prompt. For example, the present image button G162 is a button that receives an operation to add a present X-ray image to the prompt. When the present image button G162 is pressed, the prompt display functions G155 and G155b receive an operation to specify the present image to be added to the prompt.

The present and past image button G163 is a button that receives an operation to add a present image and a past image to the prompt. When the present and past image button G163 is pressed, the prompt display functions G155 and G155b receive an operation to specify the present image and the past image to be added to the prompt.

The prompt display functions G155 and G155b display prompt images G131e having the prompt corresponding to the type of additional information specified in the additional information area G16 in the prompt list area G13e. When the measurement result button G161 is pressed, for example, the prompt display functions G155 and G155b display the prompt images G131d having the prompt “let's generate a summary from the measurement results”in the prompt list area G13e.

### Fourth Modification

The medical information processing apparatuses 10, 10a, and 10b display the prompt and the generation results acquired when the prompt is input to the generative model in a manner associated with each other. This configuration enables the user to find out the generation results generated by inputting the prompt to the generative model.

The prompt input function 156 inputs one or a plurality of registered prompts identified by the degree of conformity acquired by the conformity acquisition function 153 to the generative model before the registered prompt is selected in a prompt presentation image G1f. In other words, the prompt input function 156 transmits the selected registered prompt to the generative model server 20.

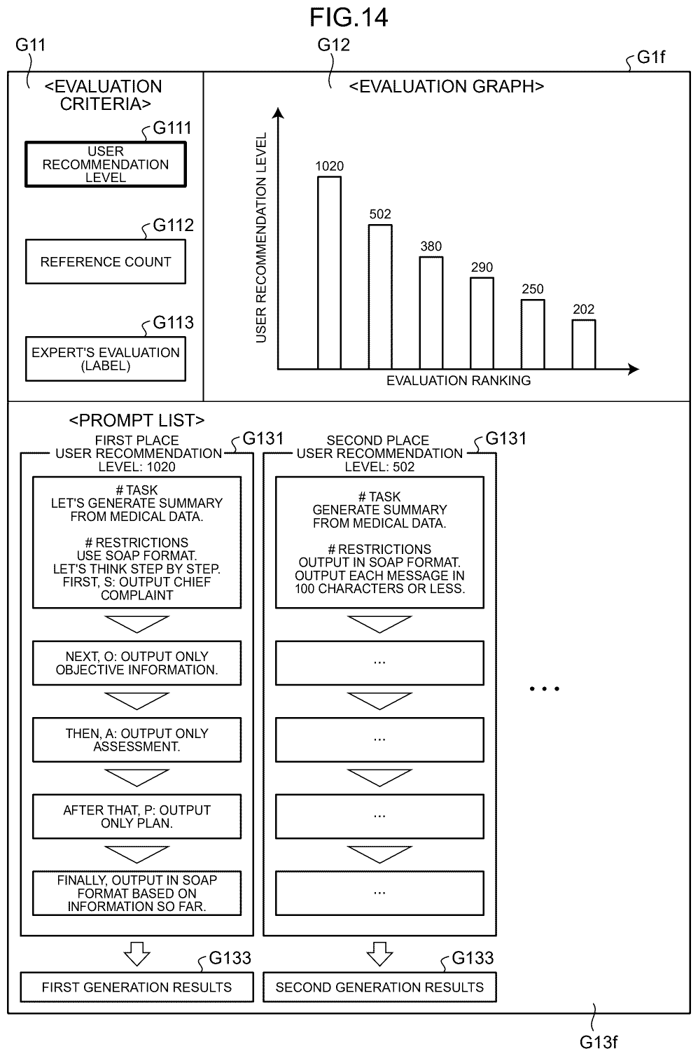

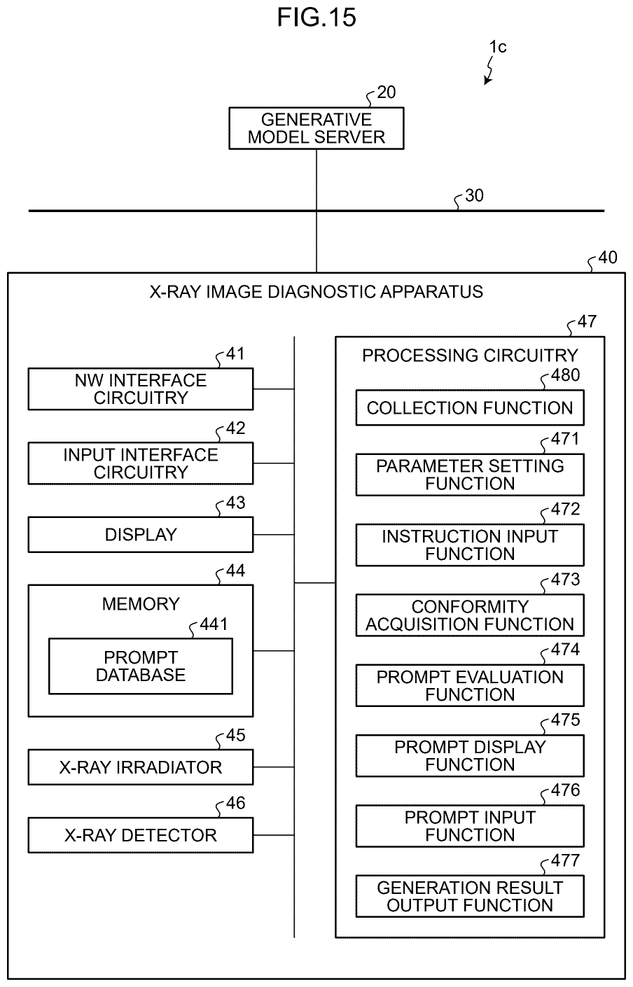

FIG. 14 is a diagram of an example of the prompt presentation image G1f. The prompt display functions G155 and G155b display the prompt presentation image G1f including the generation results generated by inputting each registered prompt to the generative model on the display 13. The prompt presentation image G1f includes the evaluation criteria area G11, the evaluation graph area G12, and a prompt list area G13f.

The evaluation criteria area G11 and the evaluation graph area G12 are the same as those illustrated in FIG. 3.

The prompt display functions G155 and G155b display the prompt image G131 and a generation result image G133 in a manner associated with each other in the prompt list area G13f. The generation result image G133 indicates the generation results generated by the generative model by inputting the registered prompt of the prompt image G131. The generation result image G133 receives an operation to select generation results from the generation results of the respective registered prompts identified by the evaluation value. The generation result image G133 is an example of an operation control unit. The prompt display functions G155 and G155b display the registered prompt corresponding to the selected generation results. For example, the prompt display functions G155 and G155b display the registered prompt in an enlarged manner. This configuration enables the user to select the registered prompt based on the generation results generated by inputting the registered prompt to the generative model.

### Fourth Embodiment

FIG. 15 is a diagram of an example of a medical information processing system 1c according to a fourth embodiment. The medical information processing system 1c may include an X-ray image diagnostic apparatus 40.

The X-ray image diagnostic apparatus 40 includes network (NW) interface circuitry 41, input interface circuitry 42, a display 43, a memory 44, an X-ray irradiator 45, an X-ray detector 46, and processing circuitry 47.

The NW interface circuitry 41 is connected to the processing circuitry 47 and controls transmission and communications of various data to and from each apparatus connected via the network 30. In other words, the NW interface circuitry 41 is an interface similar to the NW interface circuitry 11.

The input interface circuitry 42 is connected to the processing circuitry 47, converts input operations received from the user (medical worker) into electrical signals, and outputs them to the processing circuitry 47. In other words, the input interface circuitry 42 is an interface similar to the input interface circuitry 12.

The display 43 is connected to the processing circuitry 47 and displays various kinds of information and image data output from the processing circuitry 47. The display 43 is a display device similar to the display 13.

The memory 44 is connected to the processing circuitry 47 and stores therein various data. The memory 44 is a storage device similar to the memory 14. The memory 44 stores therein a prompt database 441. The prompt database 441 is a database having the same information as that of the prompt databases 141 and 141b.

The X-ray irradiator 45 outputs X-rays. More specifically, the X-ray irradiator 45 includes an X-ray tube and an X-ray diaphragm. The X-ray tube receives application of high voltage from a high-voltage generation unit and generates X-rays. The X-ray diaphragm narrows the irradiation range of the X-rays generated by the X-ray tube.

The X-ray detector 46 detects X-rays transmitted through a subject. The X-ray detector 46 is an X-ray flat panel detector (FPD) including detection elements arrayed in a matrix, for example. The X-ray detector 46 detects X-rays output from the X-ray tube and transmitted through the subject and outputs detection signals corresponding to the detected X-ray dose to the processing circuitry 47.

The processing circuitry 47 controls the entire operations of the X-ray image diagnostic apparatus 40. The processing circuitry 47 includes a collection function 480, a parameter setting function 471, an instruction input function 472, a conformity acquisition function 473, a prompt evaluation function 474, a prompt display function 475, a prompt input function 476, and a generation result output function 477, for example. In the embodiment, processing functions performed by the collection function 480, the parameter setting function 471, the instruction input function 472, the conformity acquisition function 473, the prompt evaluation function 474, the prompt display function 475, the prompt input function 476, and the generation result output function 477 serving as the components are stored in the memory 44 as computer programs executable by a computer. The processing circuitry 47 is a processor that reads each computer program from the memory 44 and executes it to implement the function corresponding to the computer program. In other words, the processing circuitry 47 that reads the computer programs include the functions in the processing circuitry 47 illustrated in FIG. 15.

The collection function 480 controls an X-ray high voltage device and adjusts the voltage supplied to the X-ray tube to control the X-ray dose with which the subject is irradiated and turning on/off. For example, the collection function 480 controls the operation of the X-ray diaphragm and adjusts the opening of the aperture blades included in a collimator, thereby controlling the irradiation range of X-rays with which the subject is irradiated. The collection function 480 also controls the operation of the X-ray diaphragm and adjusts the position of a filter, thereby controlling the irradiation range of X-rays and X-ray conditions. The collection function 480 generates an X-ray image based on the detection signals received from the X-ray detector and stores it in the memory 44.

The parameter setting function 471 has the same functions as those of the parameter setting function 151. The instruction input function 472 has the same functions as those of the instruction input function 152. The conformity acquisition function 473 has the same functions as those of the conformity acquisition function 153. The prompt evaluation function 474 has the same functions as those of the prompt evaluation functions 154 and 154b.

The prompt display function 475 has the same functions as those of the prompt display functions 155 and 155b. For example, the prompt display function 475 may display the prompt presentation image G1e illustrated in FIG. 13. In this case, the present image button G162 is a button that receives an operation to add a present X-ray image generated by the collection function 480 to the prompt, for example. The present and past image button G163 is a button that receives an operation to add the present X-ray image generated by the collection function 480 and a past X-ray image generated by the collection function 480 to the prompt.

When the present image button G162 is pressed, the prompt display function 475 displays a prompt to which the present X-ray image is added. For example, the prompt display function 475 displays a prompt that requests extraction of an abnormal region from the X-ray image. When the present and past image button G163 is pressed, the prompt display function 475 displays a prompt to which the present X-ray image and the past X-ray image are added. For example, the prompt display function 475 displays a prompt that requests extraction of differences between the present X-ray image and the past X-ray image.

The prompt input function 476 has the same functions as those of the prompt input function 156. The generation result output function 477 has the same functions as those of the generation result output function 157. The processing circuitry 47 may also include the evaluation update function 158 and the reproducibility evaluation function 159.

At least one of the embodiments and the modifications described above can output the prompt that causes the generative model to generate an answer desired by the user.

While certain embodiments have been described, these embodiments have been presented by way of example only, and are not intended to limit the scope of the inventions.

Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray image diagnostic apparatus comprising:
an X-ray irradiator configured to output an X-ray;
an X-ray detector configured to detect the X-ray output by the X-ray irradiator and transmitted through a subject; and
processing circuitry configured to:
generate an X-ray image based on detection data obtained by detection by the X-ray detector,
receive a first instruction,
receive an instruction to add the X-ray image,
acquire a degree of conformity between the first instruction and a second instruction based on a database in which a plurality of the second instructions and a plurality of prompts are associated with each other,
acquire an evaluation value obtained by evaluating the plurality of prompts identified by the acquired degree of conformity, and
display one or a plurality of prompts to which the X-ray image is added selected from the plurality of prompts based on the evaluation value.

2. A medical information processing apparatus comprising processing circuitry configured to:
receive a first instruction,
acquire a degree of conformity between the first instruction and a second instruction based on a database in which a plurality of the second instructions and a plurality of prompts are associated with each other,
acquire an evaluation value obtained by evaluating the plurality of prompts identified by the acquired degree of conformity, and
display one or a plurality of prompts selected from the plurality of prompts based on the evaluation value.

3. The medical information processing apparatus according to claim 2, wherein the processing circuitry updates the evaluation value.

4. The medical information processing apparatus according to claim 2, wherein
the processing circuitry
evaluates reproducibility between a generation result generated by a generative model by inputting the prompt and the generation result generated in past, and
displays the prompt based on the evaluation value and a value according to the reproducibility.

5. The medical information processing apparatus according to claim 4, wherein the processing circuitry displays an image indicating that the reproducibility of the prompt is lower than a threshold.

6. The medical information processing apparatus according to claim 4, wherein the processing circuitry does not display the prompt the reproducibility of which is lower than a threshold.

7. The medical information processing apparatus according to claim 2, wherein
the processing circuitry
receives specification of a type of additional information to be added to the prompt, and
displays the prompt according to the specified type of the additional information.

8. The medical information processing apparatus according to claim 2, wherein
the processing circuitry
receives an operation to select a generation result from a plurality of generation results of respective the plurality of prompts identified by the evaluation value, and
displays a prompt corresponding to the selected generation result.

9. The medical information processing apparatus according to claim 2, wherein the processing circuitry acquires the evaluation value including a user recommendation level or a reference count.

10. The medical information processing apparatus according to claim 9, wherein the user recommendation level is information indicating a number of times the prompt is displayed as a good prompt by a user.

11. The medical information processing apparatus according to claim 9, wherein the reference count is a number of times the prompt is evaluated.

12. The medical information processing apparatus according to claim 2, wherein
the processing circuitry
receives the first instruction including medical information, and
displays the prompt that requests processing of the medical information according to an instruction content indicated by the first instruction.

13. A medical information processing system comprising a medical information processing apparatus and a server including a generative model, wherein
the medical information processing apparatus comprises processing circuitry configured to:
receive a first instruction,
acquire a degree of conformity of a second instruction to the first instruction based on a database in which a plurality of the second instructions and a plurality of prompts are associated with each other,
acquire an evaluation value obtained by evaluating the plurality of prompts identified by the acquired degree of conformity,
display one or more of the prompts selected from the prompts based on the evaluation value, and
transmit the prompt selected from the displayed one or more of the prompts to the server, and
the server comprises a storage unit configured to store therein the generative model configured to perform processing based on the prompt transmitted from the medical information processing apparatus.

14. A medical information processing method comprising:
receiving a first instruction for a generative model,
acquiring the degree of conformity of a second instruction for the generative model to the first instruction based on a database in which a plurality of the second instructions and a plurality of prompts are associated with each other,
acquiring an evaluation value obtained by evaluating the plurality of prompts identified by the acquired degree of conformity, and
displaying one or more of the prompts selected from the prompts based on the evaluation value.
